(19) United States
(12) Patent Application Publication
(10) Pub. No.: US 2025/0057872 A1
(43) Pub. Date: Feb. 20, 2025

(54) INNATE IMMUNOMODULATORY EFFECT OF GINSENG BERRY POLYSACCHARIDES IN IMMUNOCOMPROMISED CONDITIONS

(71) Applicant: GANGNEUNG-WONJU NATIONAL UNIVERSITY INDUSTRY ACADEMY COOPERATION GROUP, Gangneung-si, Gangwon-do (KR)

(72) Inventors: Woo Jung PARK, Gangneung-si (KR); SangGuan YOU, Gangneung-si (KR)

(73) Assignee: GANGNEUNG-WONJU NATIONAL UNIVERSITY INDUSTRY ACADEMY COOPERATION GROUP, Gangneung-si, Gangwon-do (KR)

(21) Appl. No.: 18/724,260

(22) PCT Filed: Dec. 8, 2023
(86) PCT No.: PCT/KR2023/020172

§ 371 Date: Jun. 26, 2024

(30) Foreign Application Priority Data
Dec. 16, 2022 (KR) 10-2022-0176812

(51) Int. Cl. A61K 31/715; A61K 8/73; A61K 36/258; A61P 37/02; A61Q 19/00

(52) U.S. Cl. CPC A61K 31/715; A61K 8/73; A61K 36/258; A61P 37/02; A61Q 19/00

(57) ABSTRACT

An object of the present invention is to provide a composition for enhancing immunity comprising Korean ginseng berry polysaccharides (GBPs) as an active ingredient, and the Korean GBPs of the present invention have an immune-enhancing effect. Specifically, it was confirmed that the Korean GBPs of the present invention were administered to immunosuppressed mice induced under anticancer, immunosuppression, or infection conditions to increase spleen size and spleen indices, and to activate NK cell activity. In addition, it was confirmed that the Korean GBPs proliferated splenic lymphocytes and increased the expression of immune-related genes, and enhanced both innate and acquired immunities, and thus can be effectively used in related projects.

### TECHNICAL FIELD

An object of the present invention is to provide an innate immunomodulatory effect of ginseng berry polysaccharides in immunocompromised conditions.

### BACKGROUND ART

The immune system is an organization of cells and molecules that defends the body against infection and consists of two main subsystems of the innate immune system and the adaptive immune system. Immune responses such as immune organ indices (bone marrow, spleen, thymus) and changes in the population of immune cells (macrophages, splenocytes, neutrophils, natural killer cells) are essential mechanisms for enhancing the body's immune function. These immune responses activate a variety of immunomodulating functions by producing various cytokines (such as interferon-γ (IFN-γ), tumor necrosis factor-α (TNF-α), interleukin-2 (IL-2), and IL-6). Natural products from plant extracts that are other inflammatory mediators have the ability to stimulate the host immune system and may serve as potential therapies to substitute for chemotherapy.

The immune responses are a series of responses to exotic and endogenous substances (antigens) caused by activated immune cells. When microorganisms, including bacteria and viruses, and foreign substances of the living body are introduced into the living body, host cells secrete factors such as cytokines that cause the inflammatory responses to overcome the infection to cause the inflammatory responses. It is known that when these microorganisms and foreign substances of the living body are recognized by immune cells, the immune cells are activated, and the activated immune cells also secrete many factors causing the inflammatory responses to cause the inflammatory responses.

These immune responses are divided into innate immunity, which exists from birth, and acquired immunity, which is acquired by acquired adaptation to life, etc. Here, the innate immunity is also called ‘natural immunity,’ and reacts non-specifically to antigens and does not show any special memory function. The innate immune system includes skin, mucus tissue, and acidic stomach acid that block the invasion of antigens, phagocytes such as macrophages and polymorphonuclear leukocytes that are responsible for phagocytosis to remove invaders that have entered the body, etc. Such innate immunity actually defends most infections. On the other hand, the acquired immunity has a memory function for the first invading antigen and has the characteristic of reacting specifically and effectively eliminating the antigen when invasion occurs again.

When such an immune function is reduced, various immune diseases may occur, such as asthma, seasonal or perennial rhinitis, allergic rhinitis, conjunctivitis, atopic dermatitis, urticaria, hemolysis of red blood cells, acute glomerulonephritis, colds, chronic fatigue, and cancer. Prevention of these immune diseases is more important than treatment, but it is the reality that currently available therapeutic agents are difficult to be taken for prevention. Accordingly, there was a need for a method for preventing and treating immune dysfunction without taking immune boosters that may have side effects.

Anticancer drugs such as cyclophosphamide (CY) have been used in chemotherapy to treat cancer, autoimmune diseases, and immune-mediated diseases. However, the anticancer drugs may produce serious side effects such as cytotoxicity and oxidative stress and may damage the DNA of normal cells to cause a significant immunosuppressive condition. In such a case, it is important to recover the immunosuppressive condition, and a CY-induced immunosuppressive mouse model has been used in many previous studies to assess the immunomodulatory effects of functional materials.

Polysaccharides are a class of bioactive polymer substances with beneficial pharmacological activities and have anti-tumor, antioxidant, anti-diabetic, and immunomodulatory effects.

Korean ginseng (Panax ginseng C. A. Meyer) has been reported to exhibit immune-modulating in Korea, China, and some other East Asian countries, and ginseng berry has been reported to exhibit anticancer, anti-aging, and anti-diabetic effects.

Ginseng polysaccharides may exert immunomodulatory activities in both in vitro and in vivo systems, and crude polysaccharides from P. quinquefolius may increase macrophage phagocytosis and nitric oxide (NO) production as well as splenic lymphocyte proliferation.

Therefore, the present inventors confirmed that polysaccharides from Korean ginseng after cellulase- and α-amylase-based extraction had an immune-enhancing effect by inducing increases in spleen and thymus indices, lymphocyte proliferation, leukocyte count, and NK cell activity of CY-induced immunosuppressive mice, and then completed the present invention.

### DISCLOSURE

### Technical Problem

An object of the present invention is to provide a food composition for enhancing immunity including Korean ginseng berry polysaccharides (GBPs) as an active ingredient.

Another object of the present invention is to provide a cosmetic composition for enhancing immunity including Korean ginseng berry polysaccharides (GBPs) as an active ingredient.

Yet another object of the present invention is to provide a feed composition for enhancing immunity including Korean ginseng berry polysaccharides (GBPs) as an active ingredient.

### Technical Solution

An aspect of the present invention provides a food composition for enhancing immunity including Korean ginseng berry polysaccharides (GBPs) as an active ingredient.

Another aspect of the present invention provides a cosmetic composition for enhancing immunity including Korean ginseng berry polysaccharides (GBPs) as an active ingredient.

Yet another aspect of the present invention provides a feed composition for enhancing immunity including Korean ginseng berry polysaccharides (GBPs) as an active ingredient.

### Advantageous Effects

It was confirmed that Korean ginseng berry polysaccharides of the present invention had an immune enhancing effect. Specifically, it was confirmed that the Korean ginseng berry polysaccharides of the present invention were administered to immunosuppressed mice induced under anticancer, immunosuppression, or infection conditions to increase spleen size and spleen indices, and to activate NK cell activity. In addition, it was confirmed that the Korean ginseng berry polysaccharides proliferated splenic lymphocytes and increased the expression of immune-related genes, and enhanced both innate and acquired immunities, and thus can be effectively used in related projects.

### DESCRIPTION OF DRAWINGS

FIG. 1 is a diagram illustrating the spleen sizes of mice treated with various concentrations of Korean ginseng berry polysaccharides (GBPs).

FIG. 2 is a diagram illustrating the spleen indices of mice treated with various concentrations of Korean ginseng berry polysaccharides (GBPs).

FIG. 3 is a diagram illustrating splenocyte proliferation of mice induced with concanavalin A (Con A) or lipopolysaccharide (LPS) treated with Korean ginseng berry polysaccharides (GBPs).

FIG. 4 is a diagram illustrating NK cell activity by treatment with Korean ginseng berry polysaccharides (GBPs).

FIG. 5 is a diagram illustrating mRMA expression of IL-1B by treatment with Korean ginseng berry polysaccharides (GBPs).

FIG. 6 is a diagram illustrating mRMA expression of IL-2 by treatment with Korean ginseng berry polysaccharides (GBPs).

FIG. 7 is a diagram illustrating mRMA expression of IL-4 by treatment with Korean ginseng berry polysaccharides (GBPs).

FIG. 8 is a diagram illustrating mRMA expression of IL-6 by treatment with Korean ginseng berry polysaccharides (GBPs).

FIG. 9 is a diagram illustrating mRMA expression of TNF-α by treatment with Korean ginseng berry polysaccharides (GBPs).

FIG. 10 is a diagram illustrating mRMA expression of IFN-γ by treatment with Korean ginseng berry polysaccharides (GBPs).

FIG. 11 is a diagram illustrating mRMA expression of TLR-4 by treatment with Korean ginseng berry polysaccharides (GBPs).

FIG. 12 is a diagram illustrating mRMA expression of COX-2 by treatment with Korean ginseng berry polysaccharides (GBPs).

### BEST MODE OF THE INVENTION

Hereinafter, exemplary embodiments of the present invention will be described in detail with reference to the accompanying drawings. However, the following exemplary embodiments are presented as examples for the present invention, and when it is determined that a detailed description of well-known technologies or configurations known to those skilled in the art may unnecessarily obscure the gist of the present invention, the detailed description thereof may be omitted, and the present invention is not limited thereto. Various modifications and applications of the present invention are possible within the description of claims to be described below and the equivalent scope interpreted therefrom.

Terminologies used herein are terminologies used to properly express preferred exemplary embodiments of the present invention, which may vary according to a user, an operator's intention, customs in the art to which the present invention pertains, or the like. Accordingly, definitions of the terminologies need to be described based on contents throughout this specification. Throughout the specification, when a part “comprises” a certain component, it is meant that the part may further include other components, not excluding other components, unless explicitly described to the contrary.

The present invention provides a food composition for enhancing immunity including Korean ginseng berry polysaccharides (GBPs) as an active ingredient.

In an exemplary embodiment, the Korean ginseng berry extract is extracted with any one solvent selected from the group consisting of water, C to C4 lower alcohol, or a mixed solvent thereof, but is not limited thereto.

In an exemplary embodiment, the Korean GBPs consist of rhamnose, arabinose, mannose, glucose, and galactose, but are limited thereto.

In an exemplary embodiment, the Korean GBPs have molecular weights of 328.4 and 54.2×10³ (g/mol) but are not limited thereto.

In an exemplary embodiment, the Korean GBPs include 4.0% of rhamnose, 19.8% of arabinose, 2.2% of mannose, 27.7% of glucose, and 46.3% of galactose, but are limited thereto.

In an exemplary embodiment, the composition increases spleen size and spleen indices, but is not limited thereto.

In an exemplary embodiment, the composition increases T lymphocytes and B lymphocytes, but is not limited thereto.

In an exemplary embodiment, the composition increases NK cell activity, but is not limited thereto.

In an exemplary embodiment, the composition increases immune-related genes, but is not limited thereto.

In an exemplary embodiment, the immune-related gene is selected from the group consisting of IL-2, IL-4, IL-6, TNF-α, IFN-γ, TLR-4, and COX-2, but is not limited thereto.

In an exemplary embodiment, the immunity is reduced by anticancer drugs, immunosuppressants, or infection, but is not limited thereto.

In addition to containing the extract as the active ingredient, the food composition of the present invention may contain various flavoring agents or natural carbohydrates as additional ingredients, like conventional food compositions.

Examples of the above-described natural carbohydrates include general sugars, such as monosaccharides, such as glucose, fructose, etc.; disaccharides, such as maltose, sucrose, etc.; and polysaccharides, such as dextrin, cyclodextrin, etc., and sugar alcohols such as xylitol, sorbitol, erythritol, etc. The above-described flavoring agents may be advantageously used with natural flavoring agents (thaumatin), stevia extracts (e.g., rebaudioside A, Glycyrrhizin, etc.), and synthetic flavoring agents (saccharin, aspartame, etc.). The food composition of the present invention may be formulated in the same manner as the pharmaceutical composition to be used as a functional food or added to various foods. The foods capable of adding the composition of the present invention include, for example, beverages, meat, chocolate, foods, confectionery, pizza, ramen, other noodles, gums, candies, ice creams, alcohol beverages, vitamin complexes, health food supplements, etc.

In addition, the food composition may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, and the like, in addition to the extract as the active ingredient. In addition, the food composition of the present invention may contain pulp for preparing natural fruit juice, fruit juice beverages, and vegetable beverages.

The functional food composition of the present invention may be prepared and processed in the form of tablets, capsules, powders, granules, liquids, pills, etc. In the present invention, the ‘health functional food composition’ refers to foods prepared and processed by using raw materials or ingredients with functionality, which are useful for the human body according to Act No. 6727 on Health Functional Foods, and means foods taken for adjusting nutrients for the structures and functions of the human body or obtaining a useful effect on health applications such as physiological actions. The health functional food of the present invention may include conventional food additives, and the suitability as the food additives is determined by the specifications and standards for the corresponding item in accordance with the general rules of the Food Additive Codex, general test methods, etc., that are approved by the Food and Drug Administration, unless otherwise specified. The items disclosed in the “Food Additives Codex” may include, for example, chemical composites such as ketones, glycine, calcium citrate, nicotinic acid, cinnamic acid, etc.; natural additives such as desensitizing dye, licorice extract, crystal cellulose, Kaoliang color, guar gum, etc.; mixed formulations such as sodium L-glutamic acid formulations, noodle additive alkali agents, preservative formulations, tar color formulations, etc. For example, the health functional food in the form of tablets may formed by granulating a mixture obtained by mixing with the active ingredients of the present invention and an excipient, a binder, a disintegrant, and other additives in the conventional way, and then compression-molding the mixture by adding a lubricant and the like, or directly compression-molding the mixture. In addition, the health functional food in the form of tablets may also contain a flavor enhancer or the like as needed. In the health functional food in the form of capsules, hard capsules may be prepared by filling a mixture mixed with the active ingredient of the present invention and additives such as excipients into conventional hard capsules, and soft capsules may be prepared by filling a mixture mixed with the active ingredient of the present invention and additives such as excipients into capsule bases such as gelatin. The soft capsules may contain a plasticizer such as glycerin or sorbitol, a colorant, a preservative, and the like, if necessary. The health functional food in the form of pills may be prepared by molding a mixture mixed with the active ingredients of the present invention with an excipient, a binder, a disintegrant, etc. by conventional known methods, and may also be coated with white sugar or other coating agents or surface-coated with materials such as starch and talc, if necessary. The health functional food in the form of granules may be prepared by granulating a mixture mixed with the active ingredients of the present invention and an excipient, a binder, a disintegrant, etc. by conventional known methods and may contain a flavoring agent, a flavor enhancer, etc., if necessary.

Throughout this specification, ‘%’ used to indicate the concentration of a specific material is solid/solid (w/w) %, solid/liquid (w/v) %, and liquid/liquid (v/v) %, unless otherwise stated.

In an exemplary embodiment, the present invention provides a cosmetic composition for enhancing immunity including Korean ginseng berry polysaccharides (GBPs) as an active ingredient.

Products capable of adding the composition may include, for example, cosmetics such as various creams, lotions, toners, and essences, shampoos, rinses, cleansers, face washes, soaps, treatments, packs, beauty essences, etc.

The cosmetics of the present invention may include a composition selected from the group consisting of water-soluble vitamins, oil-soluble vitamins, polymer peptides, polymer polysaccharides, sphingolipids, and seaweed extracts.

The water-soluble vitamin may include any vitamin that can be blended with cosmetics, but preferably include vitamin B1, vitamin B2, vitamin B6, pyridoxine, pyridoxine hydrochloride, vitamin B12, pantothenic acid, nicotinic acid, nicotinic acid amide, folic acid, vitamin C, vitamin H, etc., and salts (thiamine hydrochloride, sodium ascorbate, etc.) or derivatives (sodium ascorbic acid-2-phosphate, magnesium ascorbic acid-2-phosphate, etc.) thereof are also included in the water-soluble vitamins which may be used in the present invention. The water-soluble vitamin may be obtained by conventional methods, such as microbial transformation, purification from a culture solution of a microorganism, enzymatic or chemical synthesis.

The oil-soluble vitamin may include any vitamin that can be blended with cosmetics, but preferably include vitamin A, carotene, vitamin D2, vitamin D3, vitamin E (d1-α tocopherol, d-α tocopherol, d-α tocopherol), etc., and derivatives thereof (ascorbyl palmitate, ascorbyl stearate, ascorbyl dipalmitate, DL-α tocopherol acetate, DL-α tocopherol nicotinic acid vitamin E, DL-pantothenyl alcohol, D-pantothenyl alcohol, pantothenyl ethyl ether, etc.), etc. are also included in the oil-soluble vitamins used in the present invention. The oil-soluble vitamin may be obtained by conventional methods, such as microbial transformation, purification from a culture solution of a microorganism, or enzymatic or chemical synthesis.

The polymer peptide may include any polymer peptide that can be blended with cosmetics, but preferably include collagen, hydrolyzed collagen, gelatin, elastin, hydrolyzed elastin, keratin, etc. The polymer peptide may be purified and obtained by conventional methods such as purification from a culture solution of a microorganism, enzymatic methods, or chemical synthesis, or may be usually purified and used from natural products such as the dermis of pig or cow, fibroin of silkworms, or the like.

The polymer polysaccharide may be any polymer polysaccharide that can be blended with cosmetics, but preferably include hydroxyethyl cellulose, xanthan gum, sodium hyaluronate, chondroitin sulfate or salts thereof (sodium salt, etc.). For example, chondroitin sulfate or salts thereof, etc. may generally be purified and used from mammals or fish.

The sphingolipid may be any sphingolipid that can be blended with cosmetics, but preferably include ceramide, phytosphingosine, glycosphingolipid, etc. The sphingolipid may generally be purified from mammals, fish, shellfish, yeast, or plants by conventional methods or obtained by chemical synthesis.

In addition to the essential ingredients above, the cosmetics of the present invention may contain other ingredients that may be generally blended with the cosmetics as needed. Other mixed ingredients that may be added herein may include fat ingredients, moisturizers, emollients, surfactants, organic and inorganic pigments, organic powders, UV absorbers, antiseptics, bactericides, antioxidants, plant extracts, pH regulating agents, alcohols, coloring matters, aromatics, blood flow stimulants, cooling agents, antiperspirants, purified water, etc. The fat ingredient may include ester-based fat, hydrocarbon-based fat, silicone-based fat, fluorine-based fat, animal fat, vegetable fat, etc.

The ester-based fat may include glyceryl tri2-ethylhexanoate, cetyl 2-ethylhexanoate, isopropyl myristate, butyl myristate, isopropyl palmitate, ethyl stearate, octyl palmitate, isocetyl isostearate, butyl stearate, ethyl linoleate, isopropyl linoleate, ethyl oleate, isocetyl myristate, isostearyl myristate, isostearyl palmitate, octyldodecyl myristate, isocetyl isostearate, diethyl sebacate, diisopropyl adipate, isoalkyl neopentanoate, glyceryl tricaprylate/tricaprate, trimethylolpropane tri2-ethylhexanoate, trimethylolpropane triisostearate, pentaelislitol tetra2-ethylhexanoate, cetyl caprylate, decyl laurate, hexyl laurate, decyl myristate, myristyl myristate, cetyl myristate, stearyl stearate, decyl oleate, cetyl ricinooleate, isostearyl laurate, isotridecyl myristate, isocetyl palmitate, octyl stearate, isocetyl stearate, isodecyl oleate, octyldodecyl oleate, octyldodecyl linoleate, isopropyl isostearate, cetostearyl 2-ethylhexanoate, stearyl 2-ethylhexanoate, hexyl isostearate, ethylene glycol dioctanoate, ethylene glycol dioleate, propylene glycol dicaprate, propylene glycol dicaprylate/dicaprate, propylene glycol dicaprylate, neopentyl glycol dicaprate, neopentyl glycol dioctanate, glyceryl tricaprylate, glyceryl triundecylate, glyceryl triisopalmitate, glyceryl triisostearate, octyldodecyl neopentanoate, isostearyl octanoate, octyl isononanoate, hexyldecyl neodecanoate, octyldodecyl neodecanoate, isocetyl isostearate, isostearyl isostearate, octyldecyl isostearate, polyglyceryl oleate ester, polyglyceryl isostearate ester, triisocetyl citrate, triisoalkyl citrate, triisooctyl citrate, lauryl lactate, myristyl lactate, cetyl lactate, octyldecyl lactate, triethyl citrate, acetyl triethyl citrate, acetyl tributyl citrate, trioctyl citrate, diisostearyl malate, 2-ethylhexyl hydroxystearate, di2-ethylhexyl succinate, diisobutyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stealoyl hydroxystearate, stearyl 12-stealoyl hydroxystearate, isostearyl 12-stealoyl hydroxystearate, etc.

The hydrocarbon-based fat may include squalene, liquid paraffin, α-olefin oligomer, isoparaffin, ceresin, paraffin, liquid isoparaffin, polybutene, microcrystalline wax, Vaseline, etc.

The silicone-based fat may include polymethyl silicone, methylphenyl silicone, methylcyclopolysiloxane, octamethylpolysiloxane, decamethylpolysiloxane, dodecamethylcyclosiloxane, a dimethylsiloxane/methylcetyloxysiloxane copolymer, a dimethylsiloxane/methylstealoxysiloxane copolymer, alkyl-modified silicone oil, amino-modified silicone oil, etc.

The fluorine-based fat may include perfluoropolyether, etc.

The animal or vegetable fat may include avocado oil, almond oil, olive oil, sesame oil, rice bran oil, safflower seed oil, soybean oil, corn oil, rapeseed oil, apricot seed oil, palm kernel oil, palm oil, castor oil, sunflower oil, grape seed oil, cottonseed oil, coconut oil, candlenut oil, wheat germ oil, rice germ oil, shea butter, evening primrose oil, macadamia nut oil, meadowfoam oil, egg yolk oil, beef tallow, horse oil, mink oil, orange roughy oil, jojoba oil, candelilla wax, carnauba wax, liquid lanolin, hydrogenated castor oil, etc.

The moisturizer may include a water-soluble low-molecular moisturizer, a fat-soluble molecular moisturizer, a water-soluble polymer, a fat-soluble polymer, and the like.

The water-soluble low-molecular moisturizer may include serine, glutamine, sorbitol, mannitol, sodium pyrrolidone-carboxylate, glycerin, propylene glycol, 1,3-butylene glycol, ethylene glycol, polyethylene glycol B (degree of polymerization n=2 or more), and polypropylene glycol (degree of polymerization n=2 or more), polyglycerin B (degree of polymerization n=2 or more), lactic acid, lactate, etc.

The fat-soluble low-molecular moisturizer may include cholesterol, cholesterol esters, etc.

The water-soluble polymer may include carboxyvinyl polymer, polyaspartate, tragacanth, xanthan gum, methyl cellulose, hydroxymethyl cellulose, hydroxyethyl cellulose, hydroxypropyl cellulose, carboxymethyl cellulose, water-soluble chitin, chitosan, dextrin, etc.

The fat-soluble polymer may include a polyvinylpyrrolidone/eicosene copolymer, a polyvinylpyrrolidone/hexadecene copolymer, nitrocellulose, dextrin fatty acid ester, polymer silicone, etc. The emollient may include long-chain acyl glutamic acid cholesteryl ester, hydroxystearic acid cholesteryl, 12-hydroxystearic acid, stearic acid, rosin acid, lanolin fatty acid cholesteryl ester, etc.

The surfactant may include a non-ionic surfactant, an anionic surfactant, a cationic surfactant, an amphoteric surfactant, etc.

The non-ionic surfactant may include self-emulsified monostearic acid glycerine, propylene glycol fatty acid ester, glycerine fatty acid ester, polyglycerine fatty acid ester, sorbitan fatty acid ester, polyoxyethylene (POE) sorbitan fatty acid ester, POE sorbite fatty acid ester, POE glycerine fatty acid ester, POE alkylether, POE fatty acid ester, POE hydrogenated caster oil, POE castor oil, a polyoxyethylene (POE)/polyoxypropylene (POP) copolymer, POE/POP alkylether, polyether-modified silicon, lauric acid alkanol amide, alkylamine oxide, hydrogenated soybean phospholipid, etc.

The anionic surfactant may include fatty acid soap, α-acylsulfonate, alkylsulfonate, alkylarylsulfonate, alkylnaphthalenesulfonate, alkylsulfate, POE alkylethersulfate, alkylamidesulfate, alkylphosphate, POE alkylphosphate, alkylamidephosphate, alkyloylalkyltaurate, N-acylamino acid salt, POE alkylethercarboxylate, alkylsulfosuccinate, sodium alkylsulfoacetate, acylated hydrolyzed collagen peptide salt, perfluoroalkyl phosphate ester, etc.

The cationic surfactant may include alkyltrimethyl ammonium chloride, stearyltrimethyl ammonium bromide, chloride, stearyltrimethylammonium cetostearyltrimethyl ammonium chloride, distearyldimethyl ammonium chloride, stearyldimethylbenzyl ammonium chloride, behenyltrimethyl ammonium bromide, benzalkonium chloride, diethylaminoethylamide stearate, dimethylaminopropylamide stearate, quaternary ammonium derivatives of lanolin, etc. The amphoteric surfactant may include carboxybetaine-type, amide betaine-type, sulfobetaine-type, hydroxyl sulfobetaine-type, amide sulfobetaine-type, phosphobetaine-type, aminocarboxylate-type, imidazoline derivative-type, amideamine-type amphoteric surfactants, etc.

The organic and inorganic pigments may include an inorganic pigment such as silicic acid, silica, magnesium silicate, talc, sericite, mica, kaolin, rouge, clay, bentonite, titan-coated mica, bismuth oxychloride, zirconium oxide, magnesium oxide, zinc oxide, titanium oxide, aluminum oxide, calcium sulfate, barium sulfate, magnesium sulfate, calcium carbonate, magnesium carbonate, iron oxide, ultramarine, chromium oxide, chromium hydroxide, calamine and a complex thereof; an organic pigment such as polyamide, polyester, polypropylene, polystyrene, polyurethane, vinyl resin, urea resin, phenol resin, fluorine resin, silica resin, acrylic resin, melamine resin, epoxy resin, polycarbonate resin, a divinylbenzene/styrene copolymer, silk powder, cellulose, CI Pigment Yellow, or CI Pigment Orange; and a complex pigment of the inorganic pigment and the organic pigment, etc.

The organic powder may include a metallic soap such as calcium stearate; a metal alkylphosphate such as zinc sodium cetylate, zinc laurate or calcium laurate; a polyvalent acylamino acid metal salt such as N-lauroyl-β-alanine calcium, N-lauroyl-β-alanine zinc or N-lauroyl glycine calcium; a polyvalent amide sulfonic acid metal salt such as N-lauroyl-taurine calcium or N-palmitoyl-taurine calcium; an N-acyl basic amino acid such as N-ε-lauroyl-L-lysine, N-ε-palmitoyllysine, N-α-palmitoyl omnithine, N-α-lauroylarginine, or N-α-hydrogenated tallow fatty acid acyl arginine; an N-acylpolypeptide such as N-lauroylglycylglycine; an α-amino fatty acid such as α-amino caprylic acid, or α-amino lauric acid; polyethylene, polypropylene, nylon, polymethylmethacrylate, polystyrene, a divinylbenzene/styrene copolymer, tetrafluoroethylene, etc.

The UV absorber may include para-aminobenzoic acid, ethyl-para-aminobenzoate, amyl-para-aminobenzoate, octyl-para-aminobenzoate, salicylic acid ethylene glycol, phenyl salicylate, octyl salicylate, benzyl salicylate, butylphenyl salicylate, homomentyl salicylate, benzyl cinnamate, 2-ethoxyethyl para-methoxy cinnamate, octyl para-methoxy cinnamate, mono-2-ethyl hexane glyceryl di-para-methoxy cinnamate, isopropyl para-methoxy cinnamate, a diisopropyl/diisopropyl cinnamic acid ester mixture, urocanic acid, ethyl urocanate, hydroxy methoxybenzophenone, hydroxy methoxybenzophenone sulfonic acid and slats thereof, dihydroxy methoxybenzophenone, sodium dihydroxy methoxy benzophenone disulfonate, dihydroxy benzophenone, tetrahydroxy benzophenone, 4-tert-butyl-4′-methoxy dibenzoylmethane, 2,4,6-trianilino-p-(carbo-2′-ethylhexyl-1′-oxy)-1,3,5-triazine, 2-(2-hydroxy-5-methylphenyl)benzotriazole, etc.

The bactericide may include hinokitiol, triclosan, trichlorohydroxydiphenyl ether, chlorhexidine gluconate, phenoxy ethanol, resorcin, isopropylmethylphenol, azulene, salicylic acid, zinc pyrithione, benzalkonium chloride, photosensitizer 301, sodium mononitroguaiacol, undecylenic acid, etc.

The antioxidant may include butylhydroxy anisole, propyl gallate, erythorbic acid, etc.

The pH regulating agent may include citric acid, sodium citrate, malic acid, sodium malate, fumaric acid, sodium fumarate, succinic acid, sodium succinate, sodium hydroxide, sodium dihydrogen phosphate, etc.

The alcohol may include a higher alcohol such as cetyl alcohol.

In addition, a blending ingredient which may be added herein is not limited thereto, and any ingredient may also be blended in a range that the objects and effects of the present invention are not hindered.

The cosmetics of the present invention may be prepared in the form of solutions, emulsions, viscous mixtures, or the like.

The ingredients included in the cosmetic composition of the present invention may include ingredients which are generally used in the cosmetic composition as active ingredients, and for example, include a conventional adjuvant and carrier such as a stabilizing agent, a solubilizing agent, vitamins, a pigment and an aromatic.

The cosmetic composition for skin whitening and skin anti-aging of the present invention may be prepared in any formulation which is generally prepared in the art, and examples thereof may include emulsions, creams, toners, packs, foundations, lotions, essences, hair care cosmetics, etc.

Specifically, the cosmetic composition of the present invention includes formulations of skin lotion, skin softener, skin toner, milky lotion, astringent, lotion, moisture lotion, nutritional lotion, massage cream, nutritional cream, moisture cream, hand cream, foundation, essence, nutritional essence, pack, soap, cleansing foam, cleansing lotion, cleansing cream, hair lotion, hair tonic, hair essence, hair shampoo, hair rinse, hair treatment, body lotion and body cleanser.

When the formulation of the present invention is the paste, cream, or gel, as a carrier ingredient, animal fiber, vegetable fiber, wax, paraffin, starch, tragacanth, cellulose derivatives, polyethylene glycol, silicone, bentonite, silica, talc, zinc oxide or the like may be used.

When the formulation of the present invention is the powder or spray, as the carrier ingredient, lactose, talc, silica, aluminum hydroxide, calcium silicate or polyamide powder may be used, and particularly, in the case of the spray, a propellant such as chlorofluorohydrocarbon, propane/butane or dimethyl ether may be additionally included.

When the formulation of the present invention is the solution or emulsion, as the carrier ingredient, a solvent, a solubilizing agent or an emulsifying agent may be used. For example, the carrier ingredient includes water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or sorbitan fatty acid ester.

When the formulation of the present invention is the suspension, as the carrier ingredient, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used.

When the formulation of the present invention is the surfactant-containing cleansing, as the carrier ingredient, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester, or the like may be used.

In an exemplary embodiment, the present invention provides a feed composition for enhancing immunity including Korean ginseng berry polysaccharides (GBPs) as an active ingredient.

The feed composition of the present invention is substituted with conventional antibiotics and inhibits the growth of harmful food pathogens, which can be expected to improve the health of animals, improve the weight gain and meat quality of livestock, and increase milk production and immunity. The feed composition of the present invention may be prepared in the form of fermented feed, compounded feed, pellet forms, silage, etc. The fermented feed may be prepared by fermenting an organic material by adding various microorganisms or enzymes other than the peptide of the present invention, and the compounded feed may be prepared by mixing various types of general feed with the peptide of the present invention. The pellet-form feed may be prepared by applying heat and pressure to the compounded feed, etc. in a pellet machine, and the silage may be prepared by fermenting green feed with the microorganism according to the present invention. Wet fermented feed may be prepared by collecting and transporting organic materials such as food waste, mixing excipients for sterilization and moisture control in a certain ratio, and then fermenting the mixture for at least 24 hours at a temperature suitable for fermentation to contain the moisture content in about 70%. Fermented dried feed may be prepared by adding the drying process to a wet fermented feed so as to adjust the moisture content to about 30% to 40%.

The feed composition of the present invention may further include ingredients added to conventional feed. Examples of ingredients to be added to such feed may include grain powder, meat powder, beans, etc. Here, the grain powder may be used with one or more selected from rice flour, wheat flour, barley flour, and corn flour. The meat powder may be used with meat powder obtained by powdering any one or more selected from chicken, beef, pork, and ostrich meat. The beans may be used with one or more selected from soybeans, kidney beans, peas, and black beans.

The feed composition of the present invention can be added to conventional feed components such as grain powder, meat powder, and beans, also can be supplemented with one or more selected nutrients and minerals to increase the nutritional value of the feed, and may also include one or more selected from anti-fungal agents, antioxidants, anti-coagulants, emulsifiers, and binders to prevent the deterioration of feed quality.

### Modes of the Invention

Hereinafter, the present invention will be described in more detail through Examples. However, these Examples are more specifically illustrative of the present invention, and the scope of the present invention is not limited to these Examples.

### Preparation Example 1. Animals

Six-week-old male BALB/c mice (21-23 g) were purchased from the Central Lab, Animal Inc. (Korea) and housed under standard conditions with temperature maintained at 22±2° C. and a 12-h/12-h dark/light cycle.

For at least one week before the experiments, a standard laboratory diet and water were provided to all mice. All experimental processes were approved by the Gangneung-Wonju National University Committee.

### Preparation Example 2. Korean Ginseng Berry Polysaccharides

Korean ginseng berry powder (20 g) purchased from Boeun, Chungcheong Province, Korea was extracted with 99% ethanol for 3 hours and the extract was centrifuged at 4000×g for 15 minutes and the precipitate was dried at room temperature. The dried precipitate was added with distilled water and extracted at 100° C. for 3 hours. The polysaccharide extract was added with 99% ethanol, followed by centrifugation, filtration, washing with acetone, and freeze-drying in the dried polysaccharide precipitate to produce crude polysaccharides (GBP-C). The GBP-C was fractionated using ion exchange chromatography on a DEAE Sepharose fast flow column (17-0709-01; GE Healthcare Bio-Science AB, Uppsala, Sweden), and Korean ginseng berry polysaccharides (GBPs) with molecular weights of 328.4 and 54.2×10³ (g/mol) consisting of rhamnose (4.0%), arabinose (19.8%), mannose (2.2%), glucose (27.7%), and galactose (46.3%) were prepared.

### Preparation Example 3. Immunosuppressive Treatments

In the present invention, 40 mice were randomly divided into 8 groups (n=5 per group) and variously treated according to Table 1.

Cyclophosphamide (CY) was used to induce immunosuppressive models by intraperitoneal (IP) injection at a dose of 80 mg/kg body weight (BW). Levamisole (LEV) as an immunomodulator capable of modulating cell-mediated, that is, T cell-induced immunity was used as a positive control at a dose of 40 mg/kg BW and commercial red ginseng syrup was used as a positive control at a dose of 100 mg/kg BW. In addition, ginseng was used with a commercially available product, and mice were sacrificed 24 hours after the last treatment of samples in Table 1.

TABLE 1

| | Treatment | | |
|---|---|---|---|
| | | Day 4 | |
| Group | Day 1 to 6 (orally) | to 6 (IP) | Day 7 to 10 (orally) |
| Normal | Saline | — | Saline |
| CY | Saline | CY | Saline |
| Red ginseng | Red ginseng syrup | CY | Red ginseng syrup |
| LEV | Levamisole | CY | Levamisole |
| Tr. 1 | GBP (50 mg/kg BW) | CY | GBP (50 mg/kg BW) |
| Tr. 2 | GBP (100 mg/kg BW) | CY | BP (100 mg/kg BW) |
| Tr. 3 | GBP (250 mg/kg BW) | CY | BP (250 mg/kg BW) |
| Tr. 4 | GBP (500 mg/kg BW) | CY | BP (500 mg/kg BW) |

### Preparation Example 4. Measurement of Spleen Index

The spleen was dissected and weighed. The spleen index was calculated according to Formula below:

Spleen index (mg/g)=spleen weight/body weight of mouse [Formula]

### Preparation Example 5. Preparation of Mouse Splenocytes

In order to isolate splenocytes from spleens of BALB/c mice, after weighing the weights of the spleens, the spleens of mice were placed in ice-cold PBS for splenocyte isolation. These splenocytes of mice were extracted using an RBC Lysis Buffer (eBioscience, USA). According to the manufacturer's instructions, the splenocytes were adjusted to a concentration of 2× 10⁶ cells/ml in an RPMI1640 medium (Gibco Laboratories, USA) supplemented with 1% fetal bovine serum and 1% streptomycin (100 μg/ml) and penicillin (100 IU/ml) (Welgene, Korea).

### Preparation Example 6. Measurement of Splenocyte Proliferation

EZ-Cytox Cell Viability Assay Kit (Daeillab Service, Korea) was used to measure splenic lymphocyte proliferation.

Splenocytes were seeded into a 96-well plate and cultured at 37° C. with 5% CO₂ for 1 hour. The cultured splenocytes were stimulated with 5 μg/ml of concanavalin A (Con A) as T cell mitogen or 10 μg/ml of lipopolysaccharide (LPS) as B cell mitogen. After incubation at 37° C. with 5% CO₂ for 48 hours, 25 μl of an EZ-Cytox reagent was added to each well and the plates were incubated at 37° C. with 5% CO₂ for another 1 hour. The absorbance at 450 nm was measured using a microplate reader (EL800; BioTek, USA).

Preparation Example 7. Analysis of NK Cell-Mediated Cytotoxicity Assay Splenocytes were co-cultured with or without YAC-1 cells (Korean Cell Line Bank) to obtain a 50:1 ratio of effector cells to NK-sensitive target cells. After incubation for 24 hours, the supernatant was collected after centrifugation. The activity of NK cells at 250×g was assessed for 5 minutes with a CytoTox 96 Non-Radioactive Cytotoxicity Assay Kit (Promega, USA). The absorbance at 490 nm was then measured.

### Preparation Example 8. Quantitative RT-PCR (qRT-PCR) Analysis

A Tri reagent (Molecular Research Center, Inc., USA) was used to extract total RNAs from splenic lymphocytes. Total RNAs were reverse-transcribed to cDNA using a High-Capacity cDNA Reverse Transcription Kit (Applied Biosystems, USA). To amplify the cDNA, the cDNA was applied to qRT-PCR using TB Green Premix Ex Taq II (Takara Bio Inc., Japan) on a QuantStudio 3 Flex Real-Time PCR System (Thermo Fisher Scientific, USA). Amplification conditions were as follows: initial denaturation at 95° C. for 30 seconds, followed by 40 cycles of 95° C. for 5 seconds and 60° C. for 34 seconds. Primer sequences were listed in Table 2.

TABLE 2

| | Accession | |
|---|---|---|
| Gene | No. | Primer sequences (5′→3′) |
| IL-1β | NM_008361.4 | Forward: GGGCCTCAAAGGAAAGAATC |
| | | Reverse: TACCAGTTGGGGAACTCTGC |
| IL-2 | NM_008366.3 | Forward: CCTGAGCAGGATGGAGAATT |
| | | ACA |
| | | Reverse: TCCAGAACATGCCGCAGAG |
| IL-4 | NM_021283.2 | Forward: ACAGGAGAAGGGACGCCAT |
| | | Reverse: GAAGCCCTACAGACGAGCTC |
| | | A |
| IL-6 | NM_031168.2 | Forward: AGTTGCCTTCTTGGGACTGA |
| | | Reverse: CAGAATTGCCATTGCACAAC |
| TNF-α | D84199.2 | Forward: ATGAGCACAGAAAGCATGAT |
| | | C |
| | | Reverse: TACAGGCTTGTCACTCGAAT |
| | | T |
| IFN-γ | NM_008337.3 | Forward: CTCAAGTGGCATAGATGT |
| | | Reverse: GAGATAATCTGGCTCTGCAG |
| | | GATT |
| TLR-4 | NM_021297.3 | Forward: CGCTCTGGCATCATCTTCAT |
| | | Reverse: GTTGCCGTTTCTTGTTCTTC |
| | | C |
| COX-2 | NM_011198.4 | Forward: AGAAGGAAATGGCTGCAGAA |
| | | Reverse: GCTCGGCTTCCAGTATTGAG |
| β-actin | NM_007393.5 | Forward: CCACAGCTGAGAGGGAAATC |
| | | Reverse: AAGGAAGGCTGGAAAAGAGC |

### Preparation Example 9. Statistical Analysis

The statistical analysis was evaluated using one-way analysis of variance (ANOVA) followed by Tukey's post-hoc test using Statistix 8.1 Statistics software (Statistix, USA). A significant difference between treatment groups was considered when the p-value was less than 0.05.

### Example 1. Effect of Korean GBPs on Spleen Index

In order to confirm the effects of Korean ginseng berry polysaccharides (GBP) on spleen size and spleen indices, spleen size and spleen indices were confirmed in a Levamisole (LEV) (40 mg/kg)-treated group, a red ginseng (100 mg/kg)-treated group, and groups treated with Korean ginseng berry polysaccharides (GBPs) for each of concentrations of 50, 100, 250, and 500 mg/kg BW in mice immunosuppressed by administering cyclophosphamide (CY) (80 mg/kg).

First, compared to the normal group, the CY-treated group showed decreases in both spleen size and spleen indices, while the LEV-treated group and the red ginseng-treated group had increased spleen size and indices. The Korean GBPs-treated group showed increased spleen size and indices in a concentration-dependent manner (50, 100, 250, and 500 mg/kg BW).

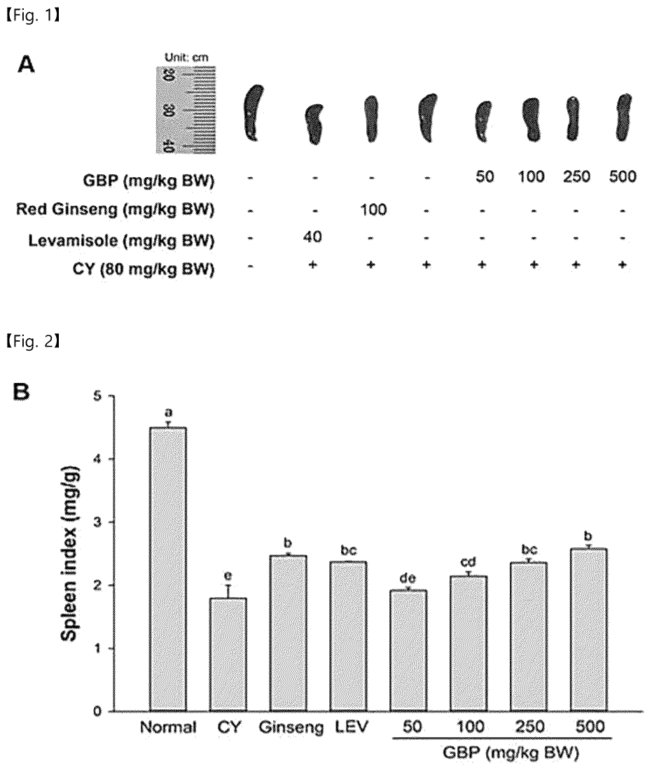

Therefore, it is suggested that the Korean GBPs of the present invention increase immunity by increasing spleen size (FIG. 1) and spleen index (FIG. 2).

### Example 2. Effect of Korean GBPs on Splenic Lymphocyte Proliferation

To determine the effect of Korean GBPs on splenic lymphocyte proliferation, the effect of Korean GBPs on splenic lymphocyte proliferation was confirmed by stimulating splenocytes with mitogen concanavalin A (Con A) as T cell mitogen or lipopolysaccharide (LPS) as B cell mitogen.

It was confirmed that the CY-treated group significantly reduced Con A- or LPS-stimulated cell proliferation (48.1% and 57.1%, respectively) compared to the normal group.

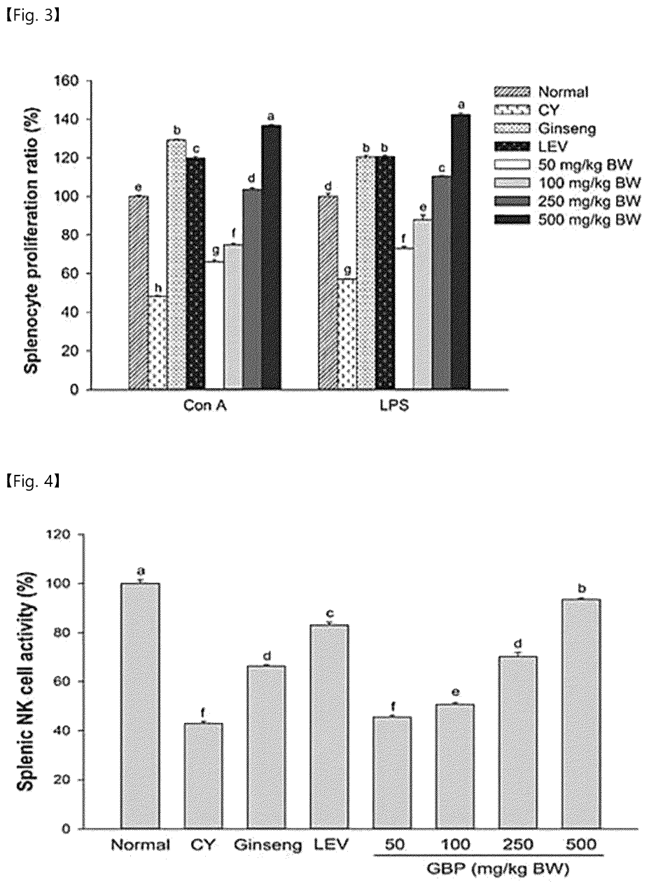

The proliferation of lymphocytes was significantly stimulated at concentrations of 250 and 500 mg/kg BW of the Korean GBPs, and splenocytes induced by concanavalin A (Con A) or lipopolysaccharide (LPS) showed high lymphocyte proliferation at a concentration of 500 mg/kg BW of Korean GBPs compared to the CY-treated group and high proliferation of T lymphocytes and B lymphocytes compared to the red ginseng-treated group and the LEV-treated group as positive control groups (FIG. 3).

Therefore, it is suggested that the Korean GBPs of the present invention increase immunity by increasing the proliferation of T lymphocytes and B lymphocytes.

### Example 3. Effect of Korean GBPs on NK Cell Activity

In order to confirm an effect of Korean GBPs on NK cell activity, a CY-treated group strongly inhibited the activity of NK cells to 42.9%.

However, it was confirmed that the NK cell activity of the Korean GBPs was significantly higher at concentrations of 100, 250, and 500 mg/kg BW than that of the CY-treated group in a concentration-dependent manner.

In addition, it was confirmed that when treated with Korean GBPs at 500 mg/kg BW, the splenic NK cell activity was recovered to a level similar to that of the normal group (FIG. 4).

Therefore, it is suggested that the Korean GBPs of the present invention increase immunity by increasing NK cell activity.

### Example 4. Effect of Korean GBPs on Immune-Related Gene Expression

To determine the effect of Korean GBPs on the expression of immune-related genes, the mRNA expression levels of immune-related genes such as IL-1β were examined by performing real-time PCR to evaluate whether the Korean GBPs may stimulate immune responses in splenic lymphocytes stimulated by mitogen concanavalin A (Con A) as T cell mitogen or lipopolysaccharide (LPS) as B cell mitogen.

The mRNAs expression levels of immune-related genes IL-2, IL-4, IL-6, TNF-α, IFN-γ, TLR-4, and COX-2 in the CY-treated group were lower than those of the normal group.

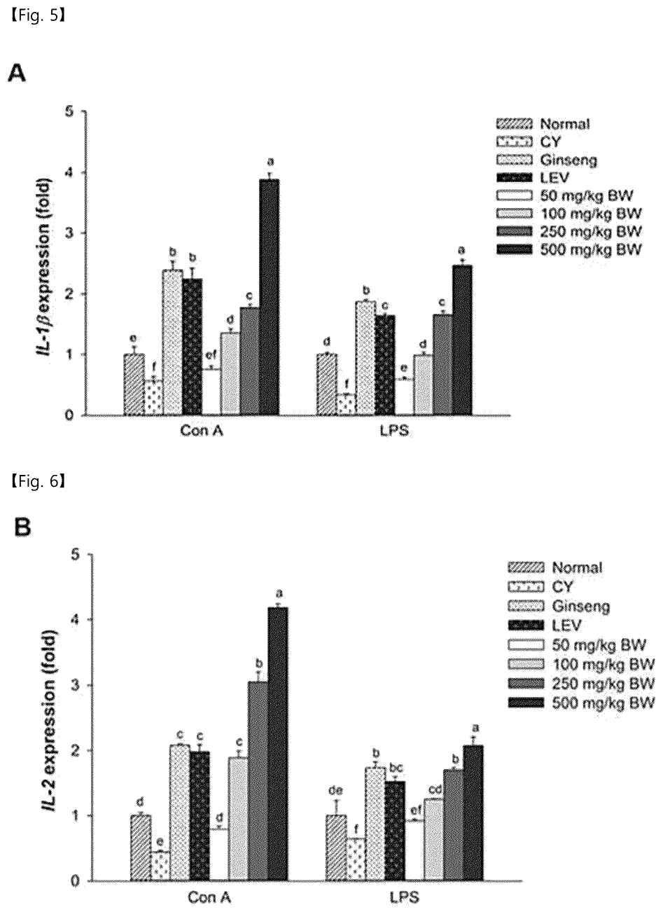

All Korean GBP-treated groups (50 to 500 mg/kg BW) showed significantly increased expression levels of immune-related genes than the CY-treated group. In the Korean GBP-treated groups, it was confirmed that the expression levels of IL-1B, IL-2, and IL-6 were significantly upregulated by concanavalin A (Con A) as T cell mitogen compared to lipopolysaccharide (LPS) as B cell mitogen (FIGS. 5, 6, and 8).

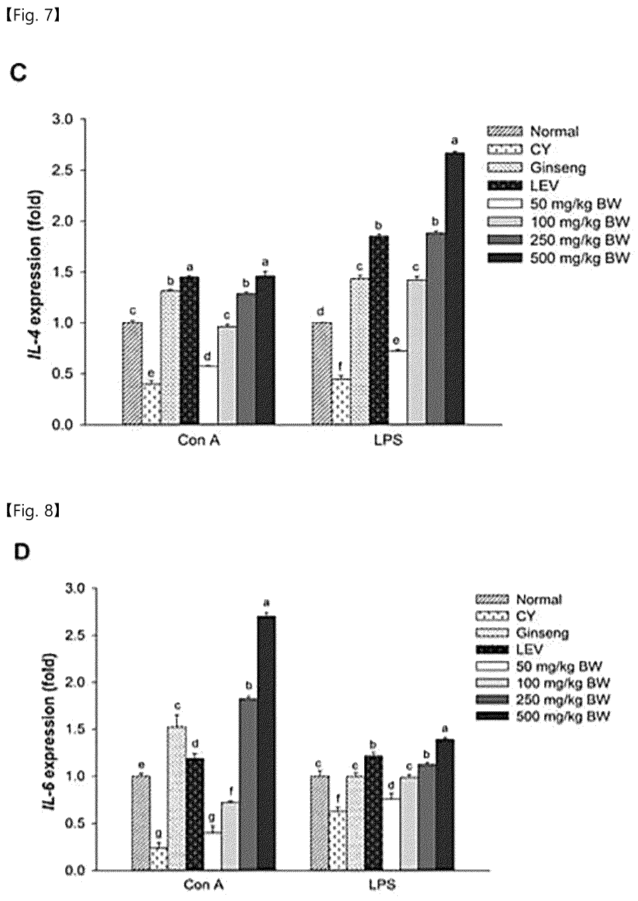

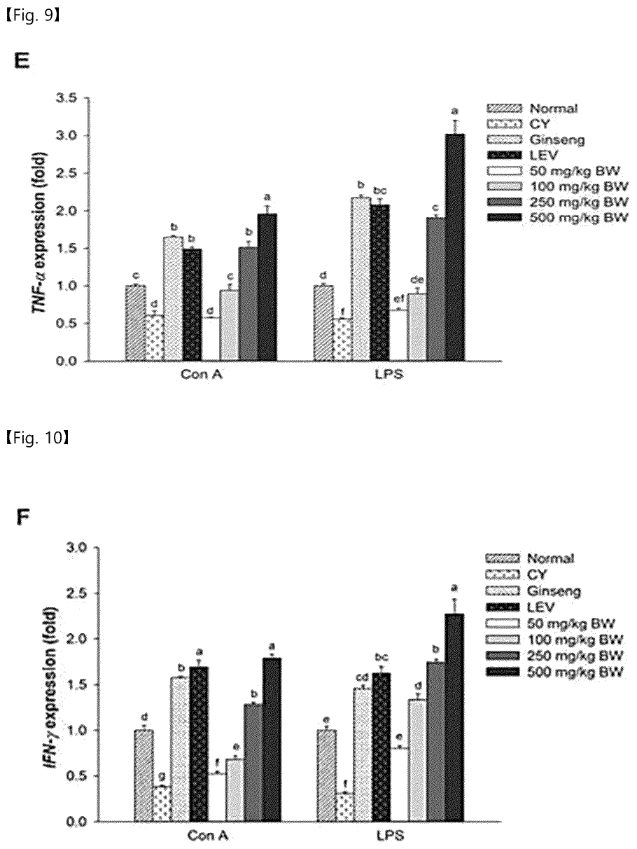

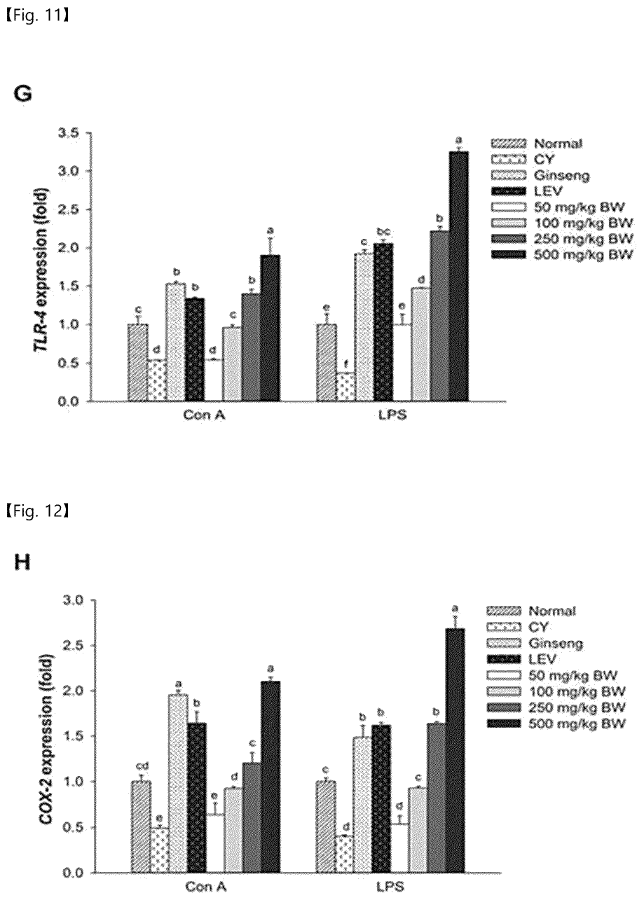

On the other hand, it was confirmed that the expression levels of IL-4, TNF-α, IFN-γ, TLR-4, and COX-2 were significantly upregulated by lipopolysaccharide (LPS) as B cell mitogen compared to T-cell mitogen (FIGS. 7, 9, 10, 11, and 12).

It was confirmed that in Korean GBPs (250 and 500 mg/kg BW), the expression levels of immune-related genes were increased compared to the normal group.

Therefore, it is suggested that the Korean GBP of the present invention increases immunity by increasing the immune-related genes.

Korean GBPs exhibited potent immunomodulatory properties in cyclophosphamide (CY)-induced immunosuppressed mice, and treatment of immunosuppressed mice with Korean GBPs improved spleen indices and enhanced immune responses.

In addition, the splenocyte proliferation, the NK cell activity, and the gene expression of immunomodulatory cytokines were upregulated. The present invention is the first invention reported that the Korean GBPs have immune-enhancing activity in an in vivo mouse model, and it is suggested that the Korean GBPs can be developed as an effective immunomodulator.

1. A method for enhancing immunity comprising administering a food composition comprising Korean ginseng berry polysaccharides (GBPs) as an active ingredient to a subject in need thereof.

2. The method of claim 1, wherein the Korean ginseng berry extract is extracted with any one solvent selected from the group consisting of water, C1 to C4 lower alcohol, and a mixed solvent thereof.

3. The method of claim 1, wherein the Korean GBPs consist of rhamnose, arabinose, mannose, glucose, and galactose.

4. The method of claim 1, wherein molecular weights of the Korean GBPs consist of 328.4 and 54.2×10³ (g/mol).

5. The method of claim 3, wherein the Korean GBPs include 4.0% of rhamnose, 19.8% of arabinose, 2.2% of mannose, 27.7% of glucose, and 46.3% of galactose.

6. The method of claim 1, wherein the subject requires increasing spleen size and spleen indices.

7. The method of claim 1, wherein the subject requires increasing T lymphocytes and B lymphocytes.

8. The method of claim 1, wherein the subject requires increasing NK cell activity.

9. The method of claim 1, wherein the subject requires increasing immune-related genes.

10. The method of claim 9, wherein the immune-related gene is selected from the group consisting of IL-2, IL-4, IL-6, TNF-α, IFN-γ, TLR-4, and COX-2.

11. The method of claim 10, wherein the immunity is reduced by anticancer drugs, immunosuppressants, or infection.

12. A method for enhancing immunity comprising applying a cosmetic composition comprising Korean ginseng berry polysaccharides (GBPs) as an active ingredient to a skin of a subject in need thereof.

13. A method for enhancing immunity comprising administering a feed composition comprising Korean ginseng berry polysaccharides (GBPs) as an active ingredient to a subject in need thereof.
